(19) United States
(12) Patent Application Publication
(10) Pub. No.: US 2004/0044007 A1
(43) Pub. Date: Mar. 4, 2004

(54) Indoline derivatives

(75) Inventors: Jan Kehler, Kgs. Lyngby (DK); Benny Bang-Andersen, Kobenhavn S (DK)

Correspondence Address: DARBY & DARBY P.C., Post Office Box 5257, New York, NY, 10150-5257, US

(73) Assignee: H. Lundbeck A/S, Valby-Copenhagen (DK)

(21) Appl. No.: 10/601,347

(22) Filed: Jun. 17, 2003

Related U.S. Application Data
(63) Continuation of application No. PCT/DK01/00835, filed on Dec. 18, 2001.

(30) Foreign Application Priority Data
Dec. 22, 2000 (DK) PA 2000 01931

(51) Int. Cl. A61K 31/55; A61K 31/496

(52) U.S. Cl. 514/254.09; 514/217.05; 514/253.09

(57) ABSTRACT

The present invention relates to methods of treating psychiatric or neurologic disorders, in particular psychoses, by administration of a compound formula of (I)

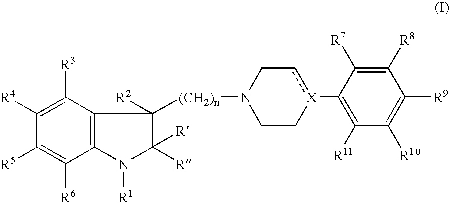

wherein R¹ is acyl, thioacyl, trifluoromethylsulfonyl or R¹ is a group R¹²SO₂—, R¹²OCO— or R¹²SCO— wherein R¹² is C₁₋₆-alkyl, C₂₋₆-alkenyl, C₂₋₆-alkynyl, C₃₋₈-cycloalkyl, C₃₋₈-cycloalkyl-C₁₋₆-alkyl or aryl, or R¹ is a group R¹³R¹⁴NCO, R¹³R¹⁴NCS—, wherein R¹³ and R¹⁴ are independently hydrogen, C₁₋₆-alkyl, C₂₋₆-alkenyl, C₂₋₆-alkynyl, C₃₋₈-cycloalkyl, C₃₋₈-cycloalkyl-C₁₋₆-alkyl or aryl, or R¹³ and R¹⁴ together with the N-atom to which they are linked form a pyrrolidinyl, piperidinyl or perhydroazepin group;

n is 1-6;

X is C, CH or N, and the dotted line emanating from X indicates a bond when X is C and no bond when X is N or CH;

R′, R″ and R² are independently selected from hydrogen and C₁₋₆-alkyl;

R³-R¹¹ are independently selected from hydrogen, halogen, cyano, nitro, C₁₋₆-alkyl, C₂₋₆-alkenyl, C₂₋₆-alkynyl, C₃₋₈-cycloalkyl, C₃₋₈-cycloalkyl-C₁₋₆-alkyl, amino, C₁₋₆-alkylamino, di-(C₁₋₆-alkyl)amino, C₁₋₆-alkylcarbonyl, aminocarbonyl, C₁₋₆-alkylaminocarbonyl, di-(C₁₋₆-alkyl)aminocarbonyl, C₁₋₆-alkoxy, C₁₋₆-alkylthio, hydroxy, trifluoromethyl, trifluoromethylsulfonyl and C₁₋₆-alkylsulfonyl; or a pharmaceutically acceptable acid addition salt thereof.

This application is a continuation of International Application No. PCT/DK01/00835, filed Dec. 18, 2001. The prior application is hereby incorporated by reference, in its entirety.

The present invention relates to a novel class of 3-indoline derivatives having affinity for the dopamine D₄ receptor. The compounds are useful in the treatment of certain psychiatric and neurologic disorders, in particular psychoses. The compounds also have affinity for the 5-HT_{2A} receptor.

### BACKGROUND OF THE INVENTION

U.S. Pat. No. 3,751,417 relates to 1-acyl-3-[2-(4-phenyl-1-piperazinyl)ethyl]indolines having the general formula

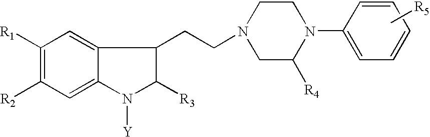

wherein R₁ is hydrogen, chloro, bromo, lower alkoxy, nitro, amino, acetamido or dimethylamino, R₂ is hydrogen, lower alkoxy or nitro, or R₁ and R₂ taken together is methylenedioxy, R₃ is hydrogen or methyl, R₄ is hydrogen or methyl, R₅ makes the phenyl-ring monosubstituted and is hydrogen, chloro, methoxy, methyl or trifluoromethyl and Y is benzoyl, p-chlorobenzoyl, p-nitrobenzoyl or lower alkanoyl. The compounds herein are said to be useful as tranquilizers and analgesics. It is known from clinical practice that tranquilizers and analgesics are generally not adequate treatment of psychoses or anxiety disorders.

U.S. Pat. No. 3,751,416 relates to similar compounds having a hydrogen in position 1 of the indoline ring. These compounds are also described as tranquilizers.

U.S. Pat. No. 5,002,948 relates to compounds having the general formula

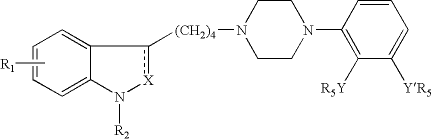

wherein R₁ is hydrogen, halogen, lower alkyl, lower alkenyl or trifluoromethyl, X is CH, CH₂, NH or CO, the dotted line indicates an optional bond, R₂ is hydrogen, lower alkyl, acyl etc., Y is O or S, Y′ is H, O, S or CH₂ and R⁵ is hydrogen, lower alkyl or alkenyl. The compounds are described as 5-HT_{1A} ligands being useful for the treatment of anxiety, depression, aggression, alcohol abuse and diseases related to the cardiovascular, the gastrointestinal and the renal system.

U.S. Pat. No. 3,900,563 relates to compounds said to be useful for the treatment of psychotic disorders. The compounds disclosed herein have the general formula

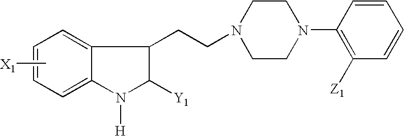

wherein X₁ is 5,6-dimethoxy or 5,6-methylendioxy, Y₁ is hydrogen or methyl and Z₁ is hydrogen or methoxy. The compounds are shown in animals at doses of 10 mg/kg to induce catalepsy predicting extrapyramidal side effects. The compounds of the present invention do not induce catalepsy at doses of 20 mg/kg.

U.S. Pat. No. 4,302,589 relates to substituted cis-2-methyl-3-[(piperazinyl) and (piperidino)ethyl]indolines having the general formula

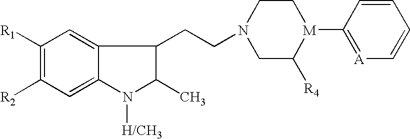

wherein R₁ is fluoro, chloro, trifluoromethyl or methoxy, R₂ is hydrogen, chloro and methoxy, and M and A are carbon or nitrogen. These compounds are described as antipsychotics.

WO 92/22554 relates to certain 4-(phenylalkyl)piperidines having affinity for sigma receptors. Nothing is said about effect at dopamine D₄ receptors.

Dopamine D₄ receptors belong to the dopamine D₂ subfamily of receptors, which is considered to be responsible for the antipsychotic effects of neuroleptics. The side effects of neuroleptic drugs which primarily exert their effect via antagonism of D₂ receptors are known to be due to D₂ receptor antagonism in the striatal regions of the brain. However, dopamine D₄ receptors are primarily located in areas of the brain other than striatum, suggesting that antagonists of the dopamine D₄ receptor will be devoid of extrapyramidal side effects. This is illustrated by the antipsychotic clozapine which exerts higher affinity for D₄ than D₂ receptors and is lacking extrapyramidal side effects (Van Tol et al. Nature 1991, 350, 610; Hadley Medicinal Research Reviews 1996, 16, 507-526 and Sanner Exp. Opin. Ther. Patents 1998, 8, 383-393).

A number of D₄ ligands which were postulated to be selective D₄ receptor antagonists (L-745,879 and U-101958) have been shown to posses antipsychotic potential (Mansbach et al. Psychopharmacology 1998, 135, 194-200). However, recently it has been reported that these compounds are partial D₄ receptor agonists in various in vitro efficacy assays (Gazi et al. Br. J. Pharmacol. 1998, 124, 889-896 and Gazi et al. Br. J. Pharmacol. 1999, 128, 613-620). Furthermore, it was shown that clozapine, which is an effective antipsychotic, is a silent antagonist (Gazi et al. Br. J. Pharmacol. 1999, 128, 613-620).

Consequently, D₄ ligands which are partial D₄ receptor agonists or antagonists may have beneficial effects against psychoses.

Dopamine D₄ antagonists may also be useful for the treatment of cognitive deficits (Jentsch et al. Psychopharmacology 1999, 142, 78-84.

It has also been suggested that dopamine D₄ antagonists may be useful to reduce dyskinesia occurring as a result of the treatment of Parkinson's disease with L-dopa (Tahar et al. Eur. J. Pharmacol. 2000, 399, 183-186).

Furthermore, evidence for a genetic association between the “primarily inattentive” subtype of attention deficit hyperactivity disorder and a tandem duplication polymorphism in the gene encoding the dopamine D₄ receptor has been published (McCracken et al. Mol. Psychiat. 2000, 5, 531-536). This clearly indicates a link between the dopamine D₄ receptor and attention deficit hyperactivity disorder and ligands affecting this receptor may be useful for the treatment of this particular disorder.

Various effects are known with respect to compounds which are ligands at the different serotonin receptor subtypes. As regards the 5-HT_{2A} receptor, which was previously referred to as the 5-HT₂ receptor, the following effects have been reported, e.g.: Antidepressive effect and improvement of the sleep quality (Meert et al. Drug. Dev. Res. 1989, 18, 119), reduction of the negative symptoms of schizophrenia and of extrapyramidal side effects caused by treatment with classical neuroleptics in schizophrenic patients (Gelders British J. Psychiatry 1989, 155 (suppl. 5), 33). Furthermore, selective 5-HT_{2A} antagonists could be effective in the prophylaxis and treatment of migraine (Scrip Report; “Migraine—Current trends in research and treatment”; PJB Publications Ltd.; May 1991) and in the treatment of anxiety (Colpart et al Psychopharmacology 1985, 86, 303-305 and Perregaard et al. Current Opinion in Therapeutic Patents 1993, 1, 101-128).

Some clinical studies implicate the 5-HT₂ receptor subtype in aggressive behaviour. Further, a typical serotonin-dopamine antagonist neuroleptics have 5-HT₂ receptor antagonistic effect in addition to their dopamine blocking properties and have been reported to possess anti-aggressive behaviour (Conner et al. Exp. Opin. Ther. Patents. 1998, 8(4), 350-351).

Recently, evidence has also accumulated, which support the rational for selective 5-HT_{2A} antagonists as drugs capable of treating positive symptoms of psychosis (Leysen et al. Current Pharmaceutical Design 1997, 3, 367-390 and Carlsson Current Opinion in CPNS Investigational Drugs 2000, 2(1), 22-24).

Accordingly, compounds with combined effects at dopamine D₄ and 5-HT_{2A} receptors may have the further benefit of improved effect on psychiatric symptoms in schizophrenic patients.

### SUMMARY OF THE INVENTION

The object of the present invention is to provide compounds which are partial agonists or antagonists at the dopamine D₄ receptor, in particular compounds with combined effects at the dopamine D₄ receptor and the 5-HT_{2A} receptor.

Thus, the present invention relates to the use of a compound having the general formula

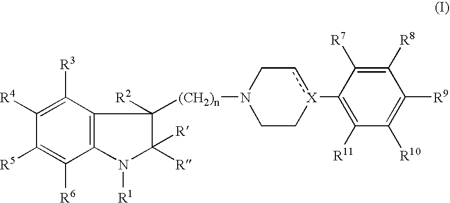

wherein R¹ is acyl, thioacyl, trifluoromethylsulfonyl, or R¹ is a group R¹²SO₂—, R¹²OCO— or R¹²SCO— wherein R¹² is C₁₋₆-alkyl, C₂₋₆-alkenyl, C₂₋₆-alkynyl, C₃₋₈-cycloalkyl, C₃₋₈-cycloalkyl-C₁₋₆-alkyl or aryl, or R¹ is a group R¹³R¹⁴NCO—, R¹³R¹⁴NCS—, wherein R¹³ and R¹⁴ are independently hydrogen, C₁₋₆-alkyl, C₂₋₆-alkenyl, C₂₋₆-alkynyl, C₃₋₈-cycloalkyl, C₃₋₈-cycloalkyl-C₁₋₆-alkyl or aryl, or R¹³ and R¹⁴ together with the N-atom to which they are linked form a pyrrolidinyl, piperidinyl or perhydroazepin group;

n is 1-6;

X is C, CH or N, and the dotted line emanating from X indicates a bond when X is C and no bond when X is N or CH;

R′, R″ and R² are independently selected from hydrogen and C₁₋₆-alkyl optionally substituted with a halogen atom; and

R³-R¹¹ are independently selected from hydrogen, halogen, cyano, nitro, C₁₋₆-alkyl, C₂₋₆-alkenyl, C₂₋₆-alkynyl, C₃₋₈-cycloalkyl, C₃₋₈-cycloalkyl-C₁₋₆-alkyl, amino, C₁₋₆-alkylamino, di-(C₁₋₆-alkyl)amino, C₁₋₆-alkylcarbonyl, aminocarbonyl, C₁₋₆-alkylaminocarbonyl, di-(C₁₋₆-alkyl)aminocarbonyl, C₁₋₆-alkoxy, C₁₋₆-alkylthio, hydroxy, trifluoromethyl, trifluoromethylsulfonyl and C₁₋₆-alkylsulfonyl;

or a pharmaceutically acceptable acid addition salt thereof, for the manufacture of a medicament useful in the treatment of positive and negative symptoms of schizophrenia, other psychoses, anxiety disorders, such as generalised anxiety disorder, panic disorder, and obsessive compulsive disorder, depression, aggression, side effects induced by conventional anti-psychotic agents, migraine, cognitive disorders, dyskinesia induced by treatment with L-dopa, attention deficit hyperactivity disorder and in the improvement of sleep quality.

The invention also relates to compounds of formula (I) as defined above, but with the proviso that

(i) R⁹ may not be hydrogen when R′, R″, R²-R⁸, R¹⁰-R¹¹ are hydrogen, n is 2 and R¹ is acetyl;

(ii) R⁹ may not be CF₃ or chloro, when R′, R″, R²-R⁸, R¹⁰-R¹¹ are hydrogen, X is C or CH, n is 2 and R¹ is acetyl;

(iii) R⁷ or R¹¹ may not be methoxy when X is N, n is 2 or 4 and R¹ is acetyl; and

(iv) R⁴ may not be methoxy.

or a pharmaceutically acceptable acid addition salt thereof.

According to a preferred embodiment, the present invention relates to the S-enantiomer of the compounds of formula (I) and the use thereof.

According to another embodiment, the present invention relates to compounds of formula (I) and the use thereof wherein R⁷ and R¹¹ are hydrogen. In a preferred embodiment, the present invention relates to such compounds of formula (I) and the use thereof wherein R¹⁰ is also hydrogen.

Another preferred group of compounds is that wherein X is CH and the dotted line is a bond.

In a particular preferred embodiment, the present invention relates to compounds wherein at least one of R⁸ and R⁹ is selected from halogen, cyano, nitro, C₁₋₆-alkyl, C₂₋₆-alkenyl, C₂₋₆-alkynyl, C₃₋₈-cycloalkyl, C₃₋₈-cycloalkyl-C₁₋₆-alkyl, amino, C₁₋₆-alkylamino, di-(C₁₋₆-alkyl)amino, C₁₋₆-alkylcarbonyl, aminocarbonyl, C₁₋₆-alkylaminocarbonyl, di-(C₁₋₆-alkyl)aminocarbonyl, C₁₋₆-alkoxy, C₁₋₆-alkylthio, hydroxy, trifluoromethyl, trifluoromethylsulfonyl and C₁₋₆-alkylsulfonyl.

In particular, R⁸ and R⁹ are identical or R⁸ is hydrogen and R⁹ is as defined above. In particular, R⁸ and R⁹ are identical and selected from halogen or alkyl, in particular methyl.

According to a more specific embodiment, the present invention relates to such compounds of formula (I) and the use thereof, wherein n is 2 or 3, preferably 2, and compounds wherein R¹ is acyl, in particular acetyl.

When R′, R″ and R² is C₁₋₆-alkyl, they are preferably methyl.

R⁴ is preferably hydrogen or halogen, in particular fluoro.

In a further embodiment, the present invention relates to compounds of formula (I) above wherein R′, R″, R², R³, R⁵ and R⁶ are hydrogen.

The compounds of the invention are partial agonists or antagonist at the dopamine D₄ receptors. The compounds also have affinity for the 5-HT_{2A} receptor.

Accordingly, the compounds of the invention are considered useful in the treatment of positive and negative symptoms of schizophrenia, other psychoses, anxiety disorders, such as generalised anxiety disorder, panic disorder and obsessive compulsive disorder, depression, aggression, side effects induced by conventional antipsychotic agents, dyskinesia induced by treatment with L-dopa, migraine, cognitive disorders, attention deficit hyperactivity disorder and in the improvement of sleep quality.

In particular, the compounds of the invention are considered useful in the treatment of positive and negative symptoms of schizophrenia without inducing extrapyramidal side effects.

In another aspect, the present invention provides a pharmaceutical composition comprising at least one compound of formula I as defined above or a pharmaceutically acceptable acid addition salt thereof in a therapeutically effective amount in combination with one or more pharmaceutically acceptable carriers or diluents.

In a further aspect, the present invention provides a method of treating the positive and negative symptoms of schizophrenia, other psychoses, anxiety disorders, such as generalised anxiety disorder, panic disorder, and obsessive compulsive disorder, depression, aggression, side effects induced by conventional anti-psychotic agents, migraine, cognitive disorders, dyskinesia induced by treatment with L-dopa, attention deficit hyperactivity disorder and in the improvement of sleep quality, comprising administration of a therapeutically acceptable amount of a compound of formula (I) as above.

### DETAILED DESCRIPTION OF THE INVENTION

The compounds of general formula I may exist as optical isomers thereof and such optical isomers as well as mixtures thereof are also embraced by the invention.

The term C₁₋₆-alkyl refers to a branched or unbranched alkyl group having from one to six carbon atoms inclusive, such as methyl, ethyl, 1-propyl, 2-propyl, 1-butyl, 2-butyl, 2-methyl-2-propyl and 2-methyl-1-propyl.

Similarly, C₂₋₆-alkenyl and C₂₋₆-alkynyl, respectively, designate such groups having from two to six carbon atoms, including one double bond and one triple bond respectively, such as ethenyl, propenyl, butenyl, ethynyl, propynyl and butynyl.

The terms C₁₋₆-alkoxy, C₁₋₆-alkylthio, C₁₋₆-alkylsulfonyl, C₁₋₆-alkylamino, C₁₋₆-alkylcarbonyl and the like designate such groups in which the alkyl group is C₁₋₆ alkyl as defined above.

The term C₃₋₈-cycloalkyl designates a monocyclic or bicyclic carbocycle having three to eight C-atoms, such as cyclopropyl, cyclopentyl, cyclohexyl, etc.

Halogen means fluoro, chloro, bromo or iodo.

As used herein the term acyl refers to a formyl, C₁₋₆-alkylcarbonyl, arylcarbonyl, aryl-C₁₋₆-alkylcarbonyl, C₃₋₈-cycloalkylcarbonyl or a C₃₋₈-cycloalkyl-C₁₋₆-alkyl-carbonyl group and the term thioacyl is the corresponding acyl group in which the carbonyl group is replaced with a thiocarbonyl group. In the term C₃₋₈-cycloalkyl-C₁₋₆-alkyl, C₃₋₈-alkyl and C₁₋₆-alkyl are as defined above.

The term aryl refers to a carbocyclic aromatic group, such as phenyl or naphthyl, in particular phenyl, which may optionally be substituted with C₁₋₆-alkyl.

The acid addition salts of the compounds of the invention are pharmaceutically acceptable salts formed with non-toxic acids. Exemplary of such organic salts are those with maleic, fumaric, benzoic, ascorbic, succinic, oxalic, bis-methylenesalicylic, methanesulfonic, ethanedisulfonic, acetic, propionic, tartaric, salicylic, citric, gluconic, lactic, malic, mandelic, cinnamic, citraconic, aspartic, stearic, palmitic, itaconic, glycolic, p-aminobenzoic, glutamic, benzenesulfonic and theophylline acetic acids, as well as the 8-halotheophyllines, for example 8-bromotheophylline. Exemplary of such inorganic salts are those with hydrochloric, hydrobromic, sulfuric, sulfamic, phosphoric and nitric acids.

The pharmaceutical compositions of this invention, or those which are manufactured in accordance with this invention, may be administered by any suitable route, for example orally in the form of tablets, capsules, powders, syrups, etc., or parenterally in the form of solutions for injection. For preparing such compositions, methods well known in the art may be used, and any pharmaceutically acceptable carriers, diluents, excipients or other additives normally used in the art may be used.

Conveniently, the compounds of the invention are administered in unit dosage form containing said compounds in an amount of 0.01 to 100 mg.

The total daily dose is usually in the range of 0.05-500 mg, and most preferably in the range of 0.1 to 50 mg of the active compound of the invention.

The compounds of the invention may be prepared as follows:

1) Alkylating a piperazine, piperidine or tetrahydropyridine of formula III with an alkylating derivative of formula II:

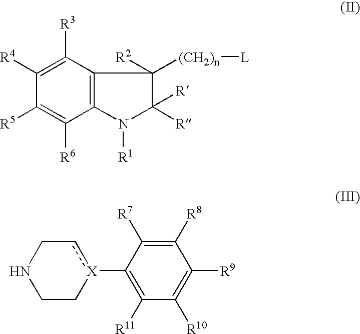

wherein R′, R″, R¹-R¹¹, X, n and the dotted line are as previously defined, and L is a leaving group such as e.g. halogen, mesylate or tosylate;

2) Reductive alkylation of an amine of formula III with a reagent of formula IV:

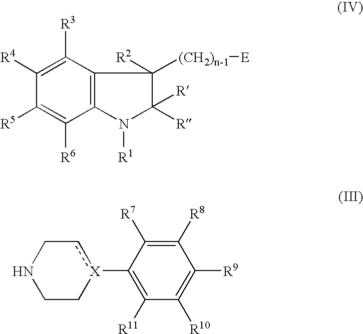

wherein R′, R″, R¹-R¹¹, X, n and the dotted line are as previously defined and E is an aldehyde or an activated carboxylic acid;

3) Reducing the double bond in the tetrahydropyridinyl ring in derivatives of formula V:

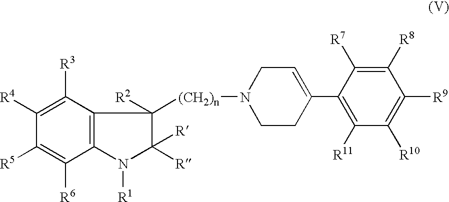

wherein R′, R″, R¹-R¹¹ and n are as previously defined; or

4) Acylating an amine of formula VI

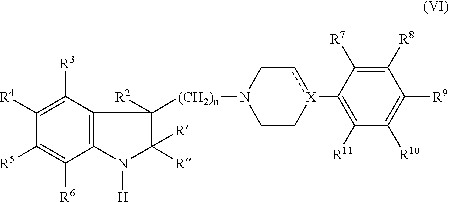

wherein R′, R″, R²-R¹¹, X, n and the dotted line are as previously defined, by the use of a carboxylic acid and a coupling reagent, an activated ester, an acid chloride, an isocyanate or by a two-step procedure by treatment with phosgene followed by addition of an amine;

whereupon the compound of formula I is isolated as the free base or a pharmaceutically acceptable acid addition salt thereof.

The alkylation according to method 1) is conveniently performed in an inert organic solvent such as a suitably boiling alcohol or ketone, preferably in the presence of an organic or inorganic base (potassium carbonate, diisopropylethylamine or triethylamine) at reflux temperature. Alternatively, the alkylation can be performed at a fixed temperature, which is different from the boiling point, in one of the above-mentioned solvents or in dimethyl formamide (DMF), dimethylsulfoxide (DMSO) or N-methylpyrrolidin-2-one (NMP), preferably in the presence of a base. The alkylating derivatives of formula II have been described in the literature (WO 98/28293), and the amines of formula III are commercially available or have been described in the literature.

The reductive alkylation according to method 2) is performed by standard literature methods. The reaction can be performed in two steps, e.g. coupling of amines of formula III with reagent of formula IV by standard methods via the carboxylic acid chloride, activated esters or by the use of carboxylic acids in combination with a coupling reagents such as e.g. dicyclohexyl carbodiimide, followed by reduction of the resulting amide with lithium aluminium hydride or alane. The carboxylic acids of formula IV can be prepared by reduction of the corresponding indolecarboxylic acids by standard methods (see e.g. WO 98/28293).

The reduction of the double bond according to method 3) is generally performed by catalytic hydrogenation at low pressure (<3 atm.) in a Parr apparatus, or by using reducing agents such as diborane or hydroboric derivatives as produced in situ from NaBH₄ in trifluoroacetic acid in inert solvents such as tetrahydrofuran (THF), dioxane or diethyl ether.

The acylation according to method 4) is conveniently performed by standard methods via the carboxylic acid chloride, activated esters or by the use of carboxylic acids in combination with coupling reagents such as e.g. dicyclohexyl carbodiimide. When the acylating reagent is carbamoyl chlorides or isocyanates, the acylation produces urea derivatives. The urea derivatives can also be prepared by a two-step procedure consisting of treatment with phosgene followed by addition of an amine.

The intermediate compounds of formula VI are prepared as described in methods 1) and 2).

Experimental Section

Melting points were determined on a Büchi SMP-20 apparatus and are uncorrected. Analytical LC-MS data were obtained on a PE Sciex API 150EX instrument equipped with IonSpray source and Shimadzu LC-8A/SLC-10A LC system. The LC conditions (C18 column 4.6×30 mm with a particle size of 3.5 μm) were linear gradient elution with water/acetonitrile/trifluoroacetic acid (90:10:0.05) to water/acetonitrile/trifluoroacetic acid (10:90:0.03) in 4 min at 2 mL/min. Purity was determined by integration of the UV trace (254 nm). The retention times, Rₜ, are expressed in minutes.

Mass spectra were obtained by an alternating scan method to give molecular weight information. The molecular ion, MH+, was obtained at low orifice voltage (5-20V) and fragmentation at high orifice voltage (100-200V).

Preparative LC-MS-separation was performed on the same instrument. The LC conditions (C18 column 20×50 mm with a particle size of 5 μm) were linear gradient elution with water/acetonitrile/trifluoroacetic acid (80:20:0.05) to water/acetonitrile/trifluoroacetic acid (5:95:0.03) in 7 min at 22.7 mL/min. Fraction collection was performed by split-flow MS detection. ¹H NMR spectra were recorded at 500.13 MHz on a Bruker Avance DRX500 instrument or at 250.13 MHz on a Bruker AC 250 instrument. Deuterated chloroform (99.8% D) or dimethyl sulfoxide (99.9% D) were used as solvents. TMS was used as internal reference standard. Chemical shift values are expressed in ppm-values. The following abbreviations are used for multiplicity of NMR signals: s=singlet, d=doublet, t=triplet, q=quartet, qui=quintet, h=heptet, dd=double doublet, dt=double triplet, dq=double quartet, tt=triplet of triplets, m=multiplet. NMR signals corresponding to acidic protons are generally omitted. Content of water in crystalline compounds was determined by Karl Fischer titration. For column chromatography silica gel of type Kieselgel 60, 230-400 mesh ASTM was used. For ion-exchange chromatography (SCX, 1 g, Varian Mega Bond Elut®, Chrompack cat. no. 220776). Prior use of the SCX-columns was pre-conditioned with 10% solution of acetic acid in methanol (3 mL).

### EXAMPLES

Preparation of Intermediates

A. Amines

4-(3,4-Dichlorophenyl)-3,6-dihydro-2H-pyridine

A mixture of butyllithium (1.6 M in hexane, 45 mL) and tetrahydrofuran (40 mL) was cooled down to −65-75° C. and subsequently added a solution of 4-bromo-1,2-dichlorobenzene (15 g) in tetrahydrofuran (25 mL). The resulting mixture was stirred at −65-75° C. for 1 h followed by the addition of ethyl 4-oxo-piperidine-1-carboxylate (11.5 g). The resulting mixture was stirred at 65-75° C. for 1 h followed by another 3 h at room temperature. The mixture was subsequently quenched by the addition of a saturated solution of ammonium chloride in water, and the aqueous phase was extracted with ethyl acetate. The combined organic extracts were dried (MgSO₄), filtered and concentrated in vacuo to give ethyl 4-(3,4-dichlorophenyl)-4-hydroxypiperidine-1-carboxylate (12.6 g). The residue was dissolved in trifluoroacetic acid (100 mL) and stirred at room temperature for 16 h. The solvent was removed in vacuo, and the residue was dissolved in a mixture of 4 M sodium hydroxide and ethanol and subsequently boiled under reflux for 48 h. The mixture was extracted with ethyl acetate, and the combined organic extracts were dried (MgSO₄), filtered and concentrated in vacuo. The residue was purified by flash chromatography on silicagel (eluent: ethyl acetate/4 M ammonia in methanol 1:1) to give the title compound (4.7 g).

4-(3,4-Dichlorophenyl)piperidine

A mixture of ethyl 4-(3,4-dichlorophenyl)-4-hydroxypiperidine-1-carboxylate (6.0 g), trifluoroacetic acid (50 mL) and triethylsilane (10 mL) was stirred at room temperature for 16 h. To the mixture was added water and ethyl acetate, and the phases were separated. The aqueous phase was extracted twice with ethyl acetate, and the combined organic extracts were dried (MgSO₄), filtered and concentrated in vacuo (5.8 g). The residue was dissolved in a mixture of 4 M sodium hydroxide and ethanol and subsequently boiled under reflux for 24 h. The mixture was extracted with ethyl acetate, and the combined organic extracts were dried (MgSO₄), filtered and concentrated in vacuo. The residue was purified by flash chromatography on silicagel (eluent: ethyl acetate/4 M ammonia in methanol 1:1) to give the title compound (1.8 g).

Preparation of the Compounds of the Invention

### Example 1

1 a, (+)-1-[2-(1-Acetyl-2,3-dihydro-1H-indol-3-yl)ethyl]-4-(3,4-dimethylphenyl)piperazine, hydrochloride.

A mixture of 1-(3,4-dimethylphenyl)piperazine (1.15 g), (+)-1-[2-(1-acetyl-2,3-dihydro-1H-indol-3-yl)ethylbromide (prepared in WO 98/28293) (1.3 g) and potassium carbonate (0.7 g) in acetonitrile (20 mL) were heated to 85° C. for 6 h. The mixture was cooled to room temperature, silicagel (7 g) added and the mixture evaporated in vacuo to give a white powder. The product was purified by flash chromatography on silicagel using as eluent ethylacetate/triethylamine (99:1). Fractions containing the product were pooled and evaporated in vacuo. The product was dissolved in tetrahydrofuran and converted to its hydrochloride by addition of HCl in diethylether (1.4 g). Mp 238-240° C. ¹H NMR (DMSO-d₆): 2.00-2.08 (m, 1H); 2.15 (s, 3H), 2.20 (s, 6H), 2.30 (m, 1H), 3.10-3.30 (m, 7H), 3.55 (m, 1H), 3.60 (m, 2H), 3.75 (m, 2H), 3.85 (m, 1H), 4.25 (m, 1H), 6.75 (d, 1H), 6.83 (s, 1H), 7.0 (t, 2H), 7.20 (t, 1H), 7.30 (d, 1H), 8.05 (d, 1H). MS m/z: 404 (MH+), 378.1.

The following compounds were prepared in a similar manner:

1b, (+)-1-[2-(1-Acetyl-2,3-dihydro-1H-indol-3-yl)ethyl]-4-(4-methylphenyl)piperazine, hydrochloride from 4-(4-methylphenyl)piperazine and (+)-1-[2-(1-acetyl-2,3-dihydro-1H-indol-3-yl)ethylbromide. Mp 217-220° C. ¹H NMR (DMSO-d₆): 2.00-2.08 (m, 1H); 2.17 (s, 3H), 2.23 (s, 3H), 2.30 (m, 1H), 3.10-3.30 (m, 7H), 3.55 (m, 1H), 3.60 (m, 2H), 3.75 (m, 2H), 3.85 (m, 1H), 4.25 (m, 1H), 6.90 (d, 2H), 7.05 (m, 3H), 7.20 (t, 1H), 7.30 (d, 1H), 8.05 (d, 1H). MS m/z: 404 (MH+), 364.0.

1c, (+)-1-[2-(1-Acetyl-2,3-dihydro-1H-indol-3-yl)ethyl]-4-(4-methylphenyl)piperidine from 4-(4-methylphenyl)piperidine and (+)-1-[2-(1-acetyl-2,3-dihydro-1H-indol-3-yl)ethylbromide. Mp 112-114° C. ¹H NMR (DMSO-d₆): 1.60-1.80 (m, 5H); 2.00 (t, 3H), 2.17 (s, 3H), 2.23 (s, 3H), 2.40 (m, 3H), 3.00 (m, 2H), 3.45 (m, 1H), 3.60 (m, 2H), 3.80 (m, 1H), 4.20 (m, 1H), 7.00 (t, 1H), 7.10 (m, 4H), 7.20 (t, 1H), 7.30 (d, 1H), 8.05 (d, 1H). MS m/z: 404 (MH+), 364.1.

1d, (+)-1-[2-(1-Acetyl-2,3-dihydro-1H-indol-3-yl)ethyl]-4-(3, 4-dichlorophenyl)piperazine, hydrochloride from 4-(3,4-dichlorophenyl)piperazine and (+)-1-[2-(1-acetyl-2,3-dihydro-1H-indol-3-yl)ethylbromide. Mp 184-186° C. ¹H NMR (DMSO-d₆): 2.00-2.08 (m, 1H); 2.15 (s, 3H), 2.30 (m, 1H), 3.10-3.30 (m, 7H), 3.55 (m, 1H), 3.60 (m, 2H), 3.75 (m, 2H), 3.85 (m, 1H), 4.25 (m, 1H), 7.0 (m, 2H), 7.20 (t, 1H), 7.25 (m, 1H), 7.30 (d, 1H), 7.43 (d, 1H), 8.05 (d, 1H). MS m/z: 404 (MH+), 417.9.

1e, (+)-]-[2-(1-Acetyl-2,3-dihydro-1H-indol-3-yl)ethyl]-4-(4-bromophenyl)piperazine, hydrochloride from 4-(4-bromophenyl)piperazine, hydrochloride and (+)-1-[2-(1-acetyl-2,3-dihydro-1H-indol-3-yl)ethylbromide. ¹H NMR (DMSO-d₆): 2.00-2.08 (m, 1H); 2.17 (s, 3H), 2.30 (m, 1H), 3.10-3.30 (m, 4H), 3.55 (m, 1H), 3.60 (m, 2H), 3.70-4.00 (m, 6H), 4.25 (m, 1H), 6.90 (d, 2H), 7.05 (t, 1H), 7.20 (t, 1H), 7.30 (d, 1H), 7.48 (d, 2H), 8.05 (d, 1H). MS m/z: 404 (MH+), 427.9.

1f,]-[2-(1-Acetyl-2,3-dihydro-1H-indol-3-yl)ethyl]-4-(3,4-dichlorophenyl)-3,6-dihydro-2H-pyridine, hydrochloride.

from 4-(3,4-dichlorophenyl)-3,6-dihydro-2H-pyridine and (+)-1-[2-(1-acetyl-2,3-dihydro-1H-indol-3-yl)ethylbromide. ¹H NMR (DMSO-d₆): 1.95-2.10 (m, 1H); 2.20 (s, 3H); 2.25-2.35 (m, 1H); 2.70-2.80 (m, 1H); 2.80-2.95 (m, 1H); 3.15-3.30 (m, 3H); 3.45-3.55 (m, 1H); 3.60-3.75 (m, 1H); 3.75-3.85 (m, 1H); 3.85-3.90 (m, 1H); 3.95-4.05 (m, 1H); 4.25 (t, 1H); 6.35 (s, 1H); 7.05 (t, 1H); 7.20 (t, 1H); 7.35 (d, 1H); 7.50 (d, 1H); 7.65 (d, 1H); 7.75 (s, 1H); 8.05 (d, 1H). MS m/z: 415 (MH+).

1 g, 1-[2-(1-Acetyl-2.3-dihydro-1H-indol-3-yl)ethyl]-4-(3.4-dichlorophenyl)piperidine, hydrochloride.

from 4-(3,4-dichlorophenyl)piperidine and (+)-1-[2-(1-acetyl-2,3-dihydro-1H-indol-3-yl)ethylbromide. ¹H NMR (DMSO-d₆): 1.95-2.35 (m, 6H); 2.20 (s, 3H); 2.80-2.95 (m, 1H); 2.95-3.25 (m, 4H); 3.50 (broad s, 1H); 3.60 (d, 2H); 3.80-3.90 (m, 1H); 4.25 (t, 1H); 7.05 (t, 1H); 7.20 (t, 1H); 7.25 (d, 1H); 7.30 (d, 1H); 7.50 (s, 1H); 7.60 (d, 1H); 8.05 (d, 1H). MS m/z: 417 (MH+).

Pharmacological Testing

The compounds of the invention were tested in well recognized and reliable tests. The tests were as follows:

Inhibition of the Binding of [³H]YM-09151-2 to D_{4.2} Receptors

By this method, the inhibition by drugs of the binding of [³H]YM-09151-2 (0.06 nM) to membranes of human cloned dopamine D_{4.2} receptors expressed in CHO-cells is determined in vitro. The method is modified from NEN Life Science Products, Inc., technical data certificate PC2533-10/96.

Inhibition of the Binding of [³H]Ketanserin to 5-HT_{2A} Receptors

The compounds were tested with respect to their affinity for 5-HT_{2A} receptors by determining their ability to inhibit binding of [³H]Ketanserin (0.50 nM) to membranes from rat brain (cortex) in vitro. Method described in Sanchez et al. Drug Dev. Res. 1991, 22, 239-250. In Table 1 below, the test results are shown:

TABLE 1
Binding Data (% inhibition of binding at 50 nM)

| | IC₅₀ (nM) or % inhib. at the | IC₅₀ (nM) at the |
|---|---|---|
| Compound | D4-receptor | 5HT2A-receptor |
| 1a | <50/88 | 5.0 |
| 1b | <50/88 | 15. |
| 1c | <50/76 | 17. |
| 1d | <50/86 | 21. |
| 1e | <50/95 | 17. |
| 1f | 13 | 27 |
| 1g | 5.4 | 21 |

The compounds of the invention have been found potently to inhibit the binding of tritiated YM-09151-2 to dopamine D₄ receptors. Further, the compounds bind potently to 5-HT_{2A} receptors.

The compounds have also been tested in a functional assay described by Gazi et al. in Br. J. Pharmacol. 1999, 128, 613-620. In this test, the compounds were shown to be partial agonists or antagonists at the dopamine D₄ receptors.

The compounds of the invention have also been tested in the following tests:

Inhibition of the Binding of [³H]Spiperone to Rat Dopamine D₂ Receptors

The compounds were tested with respect to affinity for the dopamine D₂ receptor by determining their ability to inhibit the binding of [³H]-spiperone to D₂ receptors by the method of Hyttel et al. J. Neurochem, 1985, 44, 1615.

The compounds were found to have no substantial or only weak affinity for the dopamine D₂ receptor.

The compounds of the invention containing a tetrahydropyridine ring, i.e. compounds wherein X is CH and the dotted line indicates a bond, have particularly good pharmacokinetic properties.

Thus, the compounds of the invention are considered useful in the treatment of positive and negative symptoms of schizophrenia, other psychoses, anxiety disorders, such as generalised anxiety disorder, panic disorder, and obsessive compulsive disorder, depression, side effects induced by conventional antipsychotic agents, migraine, dyskinesia induced by treatment with L-dopa, attention deficit hyperactivity disorder and in the improvement of sleep quality. In particular, the compounds of the invention are considered useful in the treatment of positive and negative symptoms of schizophrenia without inducing extrapyramidal side effects.

### FORMULATION EXAMPLES

The pharmaceutical formulations of the invention may be prepared by conventional methods in the art.

For example, tablets may be prepared by mixing the active ingredient with ordinary adjuvants and/or diluents and subsequently compressing the mixture in a conventional tabletting machine. Examples of adjuvants or diluents comprise corn starch, potato starch, talcum, magnesium stearate, gelatine, lactose, gums, and the like. Any other adjuvants or additives usually used for such purposes such as colourings, flavourings, preservatives etc. may be used provided that they are compatible with the active ingredients.

Solutions for injections may be prepared by dissolving the active ingredient and possible additives in a part of the solvent for injection, preferably sterile water, adjusting the solution to desired volume, sterilising the solution and filling it in suitable ampules or vials. Any suitable additive conventionally used in the art may be added, such as tonicity agents, preservatives, antioxidants, etc. Typical examples of recipes for the formulation of the invention are as follows:

1) Tablets containing 5.0 mg of a compound of the
invention calculated as the free base:

| | |
|---|---|
| Compound | 5.0 mg |
| Lactose | 60 mg |
| Maize starch | 30 mg |
| Hydroxypropylcellulose | 2.4 mg |
| Microcrystalline cellulose | 19.2 mg |
| Croscarmellose Sodium Type A | 2.4 mg |
| Magnesium stearate | 0.84 mg |

2) Tablets containing 0.5 mg of a compound of the
invention calculated as the free base:

| | |
|---|---|
| Compound | 0.5 mg |
| Lactose | 46.9 mg |
| Maize starch | 23.5 mg |
| Povidone | 1.8 mg |
| Microcrystalline cellulose | 14.4 mg |
| Croscarmellose Sodium Type A | 1.8 mg |
| Magnesium stearate | 0.63 mg |

3) Syrup containing per millilitre:

| | | |
|---|---|---|
| Compound | 25 | mg |
| Sorbitol | 500 | mg |
| Hydroxypropylcellulose | 15 | mg |
| Glycerol | 50 | mg |
| Methyl -paraben | 1 | mg |
| Propyl-paraben | 0.1 | mg |
| Ethanol | 0.005 | ml |
| Flavour | 0.05 | mg |
| Saccharin sodium | 0.5 | mg |
| Water | ad 1 | ml |

4) Solution for injection containing per millilitre:

| | | |
|---|---|---|
| Compound | 0.5 | mg |
| Sorbitol | 5.1 | mg |
| Acetic Acid | 0.05 | mg |
| Saccharin sodium | 0.5 | mg |
| Water | ad 1 | ml |

1. A method of treating the positive and negative symptoms of schizophrenia, other psychoses, anxiety disorders, depression, aggression, side effects induced by conventional anti-psychotic agents, migraine, cognitive disorders, dyskinesia induced by treatment with L-dopa, attention deficit hyperactivity disorder and improving sleep quality, said method comprising administering to a patient in need thereof a therapeutically effective amount of a compound of formula (I) 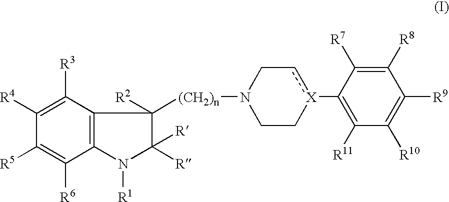
wherein R¹ is acyl, thioacyl, trifluoromethylsulfonyl, or R¹ is a group R¹²SO₂—, R¹²OCO— or R¹²SCO— wherein R¹² is C₁₋₆-alkyl, C₂₋₆-alkenyl, C₂₋₆-alkynyl, C₃₋₈-cycloalkyl, C₃₋₈-cycloalkyl-C₁₋₆-alkyl or aryl, or R¹ is a group R¹³R¹⁴NCO, R¹³R¹⁴NCS—, wherein R¹³ and R¹⁴ are independently hydrogen, C₁₋₆-alkyl, C₂₋₆-alkenyl, C₂₋₆-alkynyl, C₃₋₈-cycloalkyl, C₃₋₈-cycloalkyl-C₁₋₆-alkyl or aryl, or R¹³ and R¹⁴ together with the N-atom to which they are linked form a pyrrolidinyl, piperidinyl or perhydroazepin group;
n is 1-6;
X is C, CH or N, and the dotted line emanating from X indicates a bond when X is C and no bond when X is N or CH;
R′, R″ and R² are independently selected from hydrogen and C₁₋₆-alkyl optionally substituted with halogen; and
R³-R¹¹ are independently selected from hydrogen, halogen, cyano, nitro, C₁₋₆-alkyl, C₂₋₆-alkenyl, C₂₋₆-alkynyl, C₃₋₈-cycloalkyl, C₃₋₈-cycloalkyl-C₁₋₆-alkyl, amino, C₁₋₆-alkylamino, di-(C₁₋₆-alkyl)amino, C₁₋₆-alkylcarbonyl, aminocarbonyl, C₁₋₆-alkylaminocarbonyl, di-(C₁₋₆-alkyl)aminocarbonyl, C₁₋₆-alkoxy, C₁₋₆-alkylthio, hydroxy, trifluoromethyl, trifluoromethylsulfonyl and C₁₋₆-alkylsulfonyl; or a pharmaceutically acceptable acid addition salt thereof.

2. The method of claim 1, wherein the anxiety disorders are selected from the group consisting of generalized anxiety disorder, panic disorder and obsessive compulsive disorder.

3. The method of claim 1, wherein the compound of formula (I) is in the form of the S-enantiomer.

4. The method of claim 1 or 3 wherein R⁷ and R¹¹ are hydrogen.

5. The method of claim 4 wherein R¹⁰ is hydrogen.

6. The method of claim 1 wherein X is CH and the dotted line indicates a bond.

7. The method of claim 1 wherein at least one of R⁸ and R⁹ are independently selected from halogen, cyano, nitro, C₁₋₆-alkyl, C₂₋₆-alkenyl, C₂₋₆-alkynyl, C₃₋₈-cycloalkyl, C₃₋₈-cycloalkyl-C₁₋₆-alkyl, amino, C₁₋₆-alkylamino, di-(C₁₋₆-alkyl)amino, C₁₋₆-alkylcarbonyl, aminocarbonyl, C₁₋₆-alkylaminocarbonyl, di-(C₁₋₆-alkyl)aminocarbonyl,
C₁₋₆-alkoxy, C₁₋₆-alkylthio, hydroxy, trifluoromethyl, trifluoromethylsulfonyl and C₁₋₆-alkylsulfonyl.

8. The method of claim 1 wherein n is 2 or 3.

9. The method of claim 8 wherein n is 2.

10. The method of claim 1 wherein R¹ is acyl.

11. The method of claim 10 wherein R¹ is acetyl.

12. The method of claim 1 wherein R⁴ is hydrogen or fluoro.

13. The method of claim 1 wherein the compound of formula (I) is selected from the group consisting of
(+)-1-[2-(1-Acetyl-2,3-dihydro-1H-indol-3-yl)ethyl]-4-(3,4-dimethylphenyl)piperazine;
(+)-1-[2-(1-Acetyl-2,3-dihydro-1H-indol-3-yl)ethyl]-4-(4-methylphenyl)piperazine;
(+)-1-[2-(1-Acetyl-2,3-dihydro-1H-indol-3-yl)ethyl]-4-(4-methylphenyl)piperidine;
(+)-1-[2-(1-Acetyl-2,3-dihydro-1H-indol-3-yl)ethyl]4-(3,4-dichlorophenyl)piperazine;
(+)-1-[2-(1-Acetyl-2,3-dihydro-1H-indol-3-yl)ethyl]-4-(4-bromophenyl)piperazine;
1-[2-(1-Acetyl-2,3-dihydro-1H-indol-3-yl)ethyl]-4-(3,4-dichlorophenyl)-3,6-dihydro-2H-pyridine;
and 1-[2-(1-Acetyl-2,3-dihydro-1H-indol-3-yl)ethyl]-4-(3,4-dichlorophenyl)piperidine;
or a pharmaceutically acceptable salt thereof.

14. A 3-indoline derivative of formula (I) 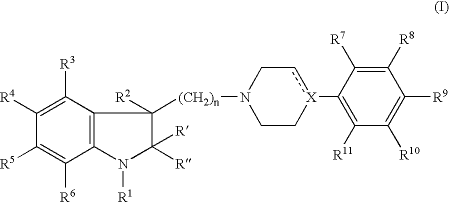
wherein R¹ is acyl, thioacyl, trifluoromethylsulfonyl, or R¹ is a group R¹²SO₂, R¹²OCO— or R¹²SCO— wherein R¹² is C₁₋₆-alkyl, C₂₋₆-alkenyl, C₂₋₆-alkynyl, C₃₋₈-cycloalkyl, C₃₋₈-cycloalkyl-C₁₋₆-alkyl or aryl, or R¹ is a group R¹³R¹⁴NCO, R¹³R¹⁴NCS—, wherein R¹³ and R¹⁴ are independently hydrogen, C₁₋₆-alkyl, C₂₋₆-alkenyl, C₂₋₆-alkynyl, C₃₋₈-cycloalkyl, C₃₋₈-cycloalkyl-C₁₋₆-alkyl or aryl, or R¹³ and R¹⁴ together with the N-atom to which they are linked form a pyrrolidinyl, piperidinyl or perhydroazepin group; and
n is 1-6;
X is C, CH or N, and the dotted line emanating from X indicates a bond when X is C and no bond when X is N or CH;
R′, R″ and R² are independently selected from hydrogen and C₁₋₆-alkyl optionally substituted with halogen;
R³-R¹¹ are independently selected from hydrogen, halogen, cyano, nitro, C₁₋₆-alkyl, C₂₋₆-alkenyl, C₂₋₆-alkynyl, C₃₋₈-cycloalkyl, C₃₋₈-cycloalkyl-C₁₋₆-alkyl, amino, C₁₋₆-alkylamino, di-(C₁₋₆-alkyl)amino, C₁₋₆-alkylcarbonyl, aminocarbonyl, C₁₋₆-alkylaminocarbonyl, di-(C₁₋₆-alkyl)aminocarbonyl, C₁₋₆-alkoxy, C₁₋₆-alkylthio, hydroxy, trifluoromethyl, trifluoromethylsulfonyl and C₁₋₆-alkylsulfonyl;
with the proviso that
(i) R⁹ may not be hydrogen when R′, R″, R²-R⁸, R¹⁰-R¹¹ are hydrogen, n is 2 and R¹ is acetyl;
(ii) R⁹ may not be CF₃ or chloro, when R′, R″, R²-R⁸, R¹⁰-R¹¹ are hydrogen, X is C or CH, n is 2 and R¹ is acetyl;
(i) R⁷ or R¹¹ may not be methoxy when X is N, n is 2 or 4 and R¹ is acetyl; and
(iv) R⁴ may not be methoxy;
or a pharmaceutically acceptable acid addition salt thereof.

15. A compound of claim 14 which is in the form of the S-enantiomer.

16. A compound of claim 14 or 15 wherein R⁷ and R¹¹ are hydrogen.

17. A compound of claim 16 wherein R¹⁰ is hydrogen.

18. A compound of claim 14 wherein X is CH and the dotted line is a bond.

19. A compound of claim 14 wherein at least one of R⁸ and R⁹ are selected from halogen, cyano, nitro, C₁₋₆-alkyl, C₂₋₆-alkenyl, C₂₋₆-alkynyl, C₃₋₈-cycloalkyl, C₃₋₈-cycloalkyl-C₁₋₆-alkyl, amino, C₁₋₆-alkylamino, di-(C₁₋₆-alkyl)amino, C₁₋₆-alkylcarbonyl, C₁₋₆-alkoxy, C₁₋₆-alkylthio, hydroxy, trifluoromethyl, trifluoromethylsulfonyl and C₁₋₆-alkylsulfonyl.

20. A compound of claim 14 wherein n is 2 or 3.

21. A compound of claim 20 wherein n is 2.

22. A compound of claim 14 wherein R¹ is acyl.

23. A compound of claim 22 wherein R¹ is acetyl.

24. A compound of claim 14 wherein R⁴ is hydrogen or fluoro and R′, R″, R², R³, R⁵ and R⁶ are hydrogen.

25. A compound of claim 14 which is selected from
(+)-1-[2-(1-Acetyl-2,3-dihydro-1H-indol-3-yl)ethyl]4-(3,4-dimethylphenyl)piperazine;
(+)-1-[2-(1-Acetyl-2,3-dihydro-1H-indol-3-yl)ethyl]-4-(4-methylphenyl)piperazine;
(+)-1-[2-(1-Acetyl-2,3-dihydro-1H-indol-3-yl)ethyl]-4-(4-methylphenyl)piperidine;
(+)-1-[2-(1-Acetyl-2,3-dihydro-1H-indol-3-yl)ethyl]-4-(3,4-dichlorophenyl)piperazine;
(+)-1-[2-(1-Acetyl-2,3-dihydro-1H-indol-3-yl)ethyl]4-(4-bromophenyl)piperazine;
1-[2-(1-Acetyl-2,3-dihydro-1H-indol-3-yl)ethyl]-4-(3,4-dichlorophenyl)-3,6-dihydro-2H-pyridine,
and 1-[2-(1-Acetyl-2,3-dihydro-1H-indol-3-yl)ethyl]-4-(3,4-dichlorophenyl)piperidine;
or a pharmaceutically acceptable salt thereof.

26. A pharmaceutical composition comprising compound of claim 14 in a therapeutically effective amount together with one or more pharmaceutically acceptable carriers or diluents.

27. A method of treating the positive and negative symptoms of schizophrenia, other psychoses, anxiety disorders, depression, aggression, side effects induced by conventional anti-psychotic agents, migraine, cognitive disorders, dyskinesia induced by treatment with L-dopa, attention deficit hyperactivity disorder and in the improvement of sleep quality, comprising administration of a therapeutically effective amount of a compound of claim 14.

28. The method of claim 27, wherein the anxiety disorders are selected from the group consisting of generalized anxiety disorder, panic disorder and obsessive compulsive disorder.
